(19) United States
(12) Patent Application Publication
(10) Pub. No.: US 2013/0243714 A1
(43) Pub. Date: Sep. 19, 2013

(54) COSMETIC MAKEUP COMPOSITION HAVING AN EXCELLENT MOISTURE-SUSTAINING CAPABILITY

(71) Applicants: Yu Jin Kang, Yongin-si (KR); Min Kyung Shim, Suwon-si (KR); Yeong Jin Choi, Yongin-si (KR); Sang Yun Kim, Yongin-si (KR)

(72) Inventors: Yu Jin Kang, Yongin-si (KR); Min Kyung Shim, Suwon-si (KR); Yeong Jin Choi, Yongin-si (KR); Sang Yun Kim, Yongin-si (KR)

(73) Assignee: AMOREPACIFIC CORPORATION, Seoul (KR)

(21) Appl. No.: 13/988,961

(22) PCT Filed: Nov. 22, 2011
(86) PCT No.: PCT/KR2011/008896

§ 371 Date: May 22, 2013

(30) Foreign Application Priority Data
Nov. 26, 2010 (KR) 10-2010-0119053

(51) Int. Cl. A61K 8/58; A61Q 1/00

(52) U.S. Cl. CPC A61K 8/585; A61Q 1/00
USPC 424/63; 556/449

(57) ABSTRACT

The present invention relates to a cosmetic makeup composition having an excellent moisture-sustaining capability, and more particularly, to a cosmetic makeup composition for providing instant moistening and moisture-sustaining capabilities when coated on the skin by stabilizing a large amount of polyol using at least one element selected from the emulsifying-agent group consisting of branched-type polyglycerine-modified dimethicones and cross-linked polymer-type polyglycerine-modified dimethicones.

### TECHNICAL FIELD

The present invention relates to a makeup cosmetic composition having excellent long-lasting moisturizing ability, and more particularly to a makeup cosmetic composition, which contains a large amount of polyol stabilized using one or more selected from the group consisting of branched polyglycerin-modified dimethicones and cross-linked polyglycerin-modified dimethicones, and thus provides immediate moisturization and has excellent long-lasting moisturizing ability when it is applied to the skin.

### BACKGROUND ART

Due to various factors, including skin aging caused by increasing age, a stress from an external environment, a severe difference in temperature between external and internal environments, frequent skin washing due to environmental pollution, and thermal skin aging caused by direct sunlight, the skin becomes dry, and the skin's horny layer is peeled off, and thus the skin looks rough, crisp, dry and dark. Thus, in order to inhibit dry skin syndromes and keratin production and impart brightness, the demand for skin moisturizers having excellent long-lasting moisturizing effects has increased.

Until now, there have been efforts to increase the moisture content of the horny layer using either humectants having the property of absorbing moisture or occlusive moisturizers for preventing moisture evaporation. Particularly, the role of moisturizers in makeup cosmetic products has become increasingly important, because these cosmetic products are applied to the skin all day long. Emulsion-type makeup cosmetic compositions have generally been prepared in the form of water-in-oil cosmetic compositions in order to increase the long-lasting moisturizing ability and immediately deliver the occlusive moisturizer oil. However, it was difficult to increase the content of the humectant polyol in such water-in-oil cosmetic compositions to a specific level or higher, because of the emulsion stability of the cosmetic compositions.

### DISCLOSURE

### Technical Problem

Accordingly, the present inventors have made efforts to find suitable emulsifiers for preparing makeup cosmetic products having excellent long-lasting moisturizing ability, and as a result, have found that a branched or crosslinked polyglycerin-modified silicone emulsifier among various kinds of emulsifiers can stabilize a large amount of polyol compared to monomeric emulsifiers or PEG-based silicone emulsifiers, thereby completing the present invention.

Therefore, it is an object of the present invention to provide a makeup cosmetic composition, which contains a large amount of stabilized polyol, and thus provides immediate moisturization and has excellent long-lasting moisturizing effects to effectively alleviate dry skin.

### Technical Solution

In order to accomplish the above object, the present invention provides a makeup cosmetic composition prepared using as an emulsifier one or more selected from the group consisting of branched polyglycerin-modified dimethicones and crosslinked polyglycerin-modified dimethicones.

### Advantageous Effects

The makeup cosmetic composition according to the present invention contains a branched or crosslinked polyglycerin-modified silicone emulsifier which can stabilize a large amount of polyol which keeps moisture from evaporating. Thus, the makeup cosmetic composition of the present invention has excellent long-lasting moisturizing ability and can alleviate dry skin.

### BEST MODE

The present invention provides a makeup cosmetic composition which contains, as an emulsifier, a large amount of polyol stabilized using one or a mixture of two or more selected from among branched polyglycerin-modified silicones and crosslinked polyglycerin-modified silicones. Thus, when the composition of the present invention is applied to the skin, it provides immediate moisturization and has excellent long-lasting moisturizing ability.

Hereinafter, the present invention will be described in further detail.

In the present invention, a polyglycerin-modified silicone emulsifier may be used as an emulsifier. Preferably, one or a mixture of two or more selected from among branched polyglycerin-modified silicones and crosslinked polyglycerin-modified silicones may be used as an emulsifier.

The branched polyglycerin-modified silicone that is used in the present invention has a structure of the following formula 1 or 2, and specific examples thereof include, but are not limited to, polyglyceryl-3 disiloxane dimethicone (trade name: KF-6100), polyglyceryl-3 polydimethyldisiloxyethyl dimethicone (trade name: KF-6104) and lauryl polyglyceryl-3 polydimethyldisiloxyethyl dimethicone (trade name: KF-6105), which are sold by Shin Etsu Chemical Co., Ltd.:

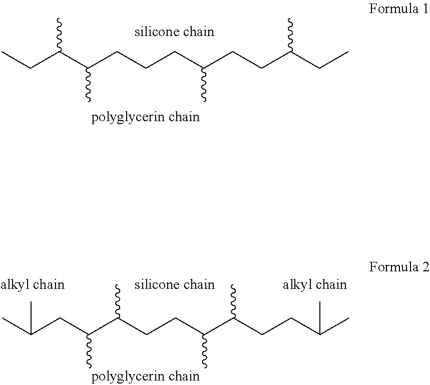

The crosslinked polyglycerin-modified silicone that is used in the present invention has a structure of the following formula 3 or 4, and specific examples thereof include, but are not limited to, dimethicone/polyglycerin-3 crosspolymer (trade name: KSG-710) and lauryl dimethicone/polyglycerin-3 crosspolymer (trade name: KSG-810, KSG-820, KSG-830 or KSG-810), which are sold by Shin Etsu Chemical Co., Ltd.:

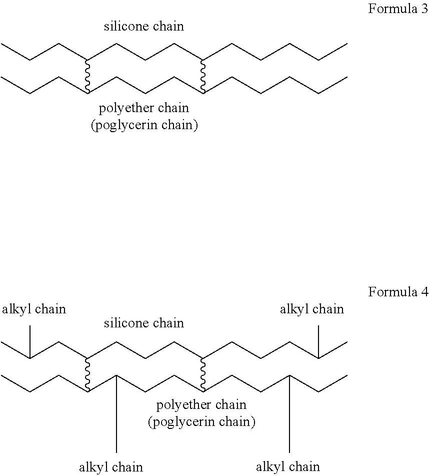

The makeup cosmetic composition according to the present invention preferably contains the emulsifier in an amount of 0.1-20 wt % based on the total weight of the composition. If the content of the emulsifier is less than 0.1 wt %, the emulsion will be very unstable, and if the content of the emulsifier is more than 20.0 wt %, the emulsion will have poor touch feeling, because it is slippery and sticky.

In addition, the makeup cosmetic composition according to the present invention may further contain, as a moisturizer, one or more polyols selected from the group consisting of glycerin, butylene glycol, propylene glycol, dipropylene glycol, pentylene glycol, hexylene glycol, polyethylene glycol, and sorbitol. The polyol may be contained in an amount of 10-80 wt %, and preferably 20-40 wt %, based on the total weight of the composition. If the content of the polyol is less than 10 wt %, the long-lasting moisturizing ability of the composition will not be satisfactory, and if the content of the polyol is more than 80 wt %, the composition will have poor touch feeling, because it is sticky and slippery.

In addition, the composition of the present invention may further contain skin-activating components or various oils known in the art, and the content of such oils is preferably 1-50 wt % based on the total weight of the composition. Examples of the oils include, but are not limited to, silicone oils such as dimethicone or cyclomethicone; ester-based oils such as cetyl ethyl hexanoate or C₁₂₋₁₅ alkyl benzoate; hydrocarbon-based oils such as hydrogenated polydecene or squalane; and mixtures thereof.

In addition, the makeup cosmetic composition according to the present invention may further contain additives, including pigments, fragrances, preservatives and thickeners, which are generally used in the preparation of makeup cosmetic products, in an amount of 0-20 wt % based on the total weight of the composition.

The formulation of the makeup cosmetic composition of the present invention is not specifically limited. For example, the composition of the present invention may be formulated as a water-in-oil or oil-in-water makeup base or foundation containing a pigment, but is not limited thereto.

### MODE FOR INVENTION

Hereinafter, the present invention will be described in further detail with reference to examples and test examples. It is to be understood, however, that these examples and test examples are for illustrative purposes only and are not intended to limit the scope of the present invention. In addition, those skilled in the art will appreciate that various modifications, additions and substitutions are possible, without departing from the scope and spirit of the present invention.

### Examples 1 to 3 and Comparative Examples 1 to 5

According to the compositions shown in Tables 1 and 2 below, water-in-oil makeup cosmetic compositions of Examples 1 to 3 and Comparative Examples 1 to 5 were prepared (unit: wt %). In Examples 1 to 3 of the present invention, lauryl polyglyceryl-3 polydimethyldisiloxyethyl dimethicone, which is a branched polyglycerin-modified silicone emulsifier, and dimethicone/polyglycerin-3 crosspolymer which is a crosslinked polyglycerin-modified silicone emulsifier were used alone or in combination. In Comparative Example 1 and 2, lauryl PEF-9 polydimethylsiloxyethyl dimethicone, which is a PEG-based silicone emulsifier, and dimethicone/PEG-10/15 crosspolymer which is a crosslinked PEG-based silicone emulsifier were used, and in Comparative Example 3, a monomeric emulsifier was used. In Comparative Examples 4 and 5, the same emulsifier used in Example 3 was used while the content of polyol was changed. The polyol used was prepared using glycerin and butylene glycol.

TABLE 1

| Components | Raw material name | Example 1 | Example 2 | Example 3 |
|---|---|---|---|---|
| Oil phase | Cyclopentasiloxane | 20.00 | 20.00 | 20.00 |
| components | Cetylethylhexanoate | 10.00 | 10.00 | 10.00 |
| | Lauryl polyglyceryl-3 | 4.00 | — | 2.00 |
| | polydimethyldisiloxy- | | | |
| | ethyl dimethicone | | | |
| | Dimethicone/ | — | 4.00 | 2.00 |
| | polyglycerin-3 | | | |
| | crosspolymer | | | |
| | Disteary ammonium | 1.00 | 1.00 | 1.00 |
| | hectorite | | | |
| | Paraoxybenzoic | q.s. | q.s. | q.s. |
| | acid ester | | | |
| Pigments | Titanium oxide and | 10.00 | 10.00 | 10.00 |
| | iron oxide | | | |
| Aqueous | Purified water | Balance | Balance | Balance |
| phase | Glycerin | 20.00 | 20.00 | 20.00 |
| components | Butylene glycol | 20.00 | 20.00 | 20.00 |
| | Disodium EDTA | 0.05 | 0.05 | 0.05 |
| | Sodium chloride | 1.00 | 1.00 | 1.00 |
| | Phenoxyethanol | q.s. | q.s. | q.s. |

TABLE 2

| | | Comparative | Comparative | Comparative | Comparative | Comparative |
|---|---|---|---|---|---|---|
| Components | Raw material name | Example 1 | Example 2 | Example 3 | Example 4 | Example 5 |
| Oil phase | Cyclopentasiloxane | 20.00 | 20.00 | 20.00 | 20.00 | 20.00 |
| components | Cetylethylhexanoate | 10.00 | 10.0 | 10.00 | 10.00 | 10.00 |
| | Lauryl polyglyceryl-3 | — | — | — | 2.00 | 2.00 |
| | polydimethyldisiloxyethyl dimethicone | | | | | |
| | Dimethicone/polyglycerin-3 | — | 2.00 | — | 2.00 | 2.00 |
| | crosspolymer | | | | | |
| | Lauryl PEG-9 polydimethylsiloxyethyl | 2.00 | — | — | — | — |
| | dimethicone | | | | | |
| | Dimethicone/PEG-10/15 crosspolymer | 2.00 | 2.00 | — | — | — |
| | Polyglyceryl-2 dipolyhydroxystearate | — | — | 4.00 | — | — |
| | (monomeric emulsifier) | | | | | |
| | Distearyl ammonium hectorite | 1.00 | 1.00 | 1.00 | 1.00 | 1.00 |
| | Paraoxybenzoic acid ester | q.s. | q.s. | q.s. | q.s. | q.s. |
| Pigments | Titanium oxide and iron oxide | 1.00 | 10.00 | 1.00 | 10.00 | 10.00 |
| Aqueous phase | Purified water | Balance | Balance | Balance | Balance | Balance |
| components | Glycerin | 20.00 | 20.00 | 20.00 | 250 | 450 |
| | Butylene glycol | 20.00 | 20.00 | 20.00 | 250 | 450 |
| | Disodium EDTA | 0.05 | 0.05 | 0.05 | 0.05 | 0.05 |
| | Sodium chloride | 1.00 | 1.00 | 1.00 | 1.00 | 1.00 |
| | Phenoxyethanol | q.s. | q.s. | q.s. | q.s. | q.s. |

Preparation Method

1) The oil phase components and pigments in the above Tables were mixed and dispersed.

2) The aqueous phase components were mixed with each other.

3) The solution of step 2) was added to and mixed with the solution of step 1), and the mixture was completely degassed, thereby preparing makeup cosmetic compositions.

The formulation stabilities, long-lasting moisturizing properties and brightness of the makeup cosmetic compositions prepared in Examples 1 to 3 and Comparative Examples 1 to 5 were evaluated in the following manner.

### Test Example 1

### Comparison of Stability

In order to examine the formulation stability of the makeup cosmetic composition of the present invention, 10 g of each of the compositions of Examples 1 to 3 and Comparative Examples 1 to 5 was taken and placed in each of a room-temperature incubator, a 55° C. incubator and a −10˜45° C. cycling chamber, and then the stabilities thereof were observed for 1 week. The results of the observation are shown in Table 3 below.

TABLE 3

| | Room- | High- | |
|---|---|---|---|
| Test | temperature | temperature | Cycling |
| material | stability | stability | stability |
| Example 1 | ∘ | Δ | ∘ |
| Example 2 | Δ | Δ | ∘ |
| Example 3 | ∘ | ∘ | ∘ |
| Comparative | Δ | x | Δ |
| Example 1 | | | |
| Comparative | x | x | x |
| Example 2 | | | |
| Comparative | x | x | x |
| Example 3 | | | |
| Comparative | ∘ | ∘ | ∘ |
| Example 4 | | | |
| Comparative | ∘ | ∘ | ∘ |
| Example 5 | | | |

<Criteria for evaluation of stability>
x: very poor (separated within one week)
Δ: slightly poor (separated within one month)
∘: good (stable over one month or longer)

As can be seen from the results in Table 3 above, the makeup cosmetic compositions of Examples 1 to 3 and Comparative Examples 4 and 5, which contain the emulsifier according to the present invention, showed excellent formulation stability compared to the compositions of Comparative Examples 1 to 3 which contain other emulsifiers.

This appears to be because the hydrophilic glycerin group of the polyglycerin-modified silicone emulsifier used in the present invention was chemically similar to the polyol of the inner phase, and thus could hold the polyol by a strong bonding force so as to stabilize a large amount of the polyol, compared to the PEG-based silicone emulsifier. Particularly, in Comparative Example 2, because only the crosslinked emulsifier was used, emulsion particles at least five times larger than conventional emulsion particles were prepared, and thus when a large amount of the polyol was contained in the inner phase, it made the interface unstable so that the emulsion particles were immediately broken under the high-temperature and cycling conditions. In Comparative Example 3, because only the monomeric emulsifier was used, it did not sufficiently hold the silicone oil used as a main oil and had weak emulsifying ability, and thus the emulsion particles were immediately broken.

In addition, in Example 3 in which both the branched polyglycerin-modified emulsifier and the crosslinked polyglycerin-modified emulsifier were used, the two emulsifiers having different sizes formed a denser interfacial film, and thus the composition had better stability than the compositions of Examples 1 and 2 in which only one kind of emulsifier was used.

### Test Example 2

### Evaluation of Long-Lasting Skin Moisturizing Ability

In order to examine the long-lasting skin moisturizing ability according to the content of polyol when each of the compositions was applied once, each of the makeup cosmetic compositions of Examples 1 to 3 and Comparative Examples 4 and 5 was applied to the face and the inside of the forearm of eighty 30-40-year-old men and women who complained of dry skin or believed to have dry skin. After 24 hours, the skin moisture content of each subject was measured using a corneometer (CM820 courage Khazaka electronic GmbH, Germany). Before the start of the application, the skin moisture content of each subject was measured using a corneometer under constant temperature and constant humidity conditions (24° C. and 40% humidity) and used as a reference value, and the changes in the skin moisture content immediately after the application and at hours, 4 hours and 8 hours after the application were measured. The results of the measurement are shown in Table 4 below.

Measurement Method Using Corneometer

Principle: moisturizing ability was determined by measuring the degree of moisture ions in the epidermis of the skin using a sensor, expressing the measurement result as a numerical value, and calculating the moisture content.

1) The corneometer probe was placed on the skin portion to be measured.

2) When the probe was pressed against the skin, the capacitance of the skin was displayed as a numerical value on a screen through a sensor.

3) After one measurement, the sensor was wiped with paper such as Kimwipe, and then another portion was measured.

TABLE 4

| | Skin moisture content (%) | | | | |
|---|---|---|---|---|---|
| | | Immediately | | | |
| Test | Before | after | After 2 | After 4 | After 8 |
| material | application | application | hours | hours | hours |
| Example 1 | 34 | 48 | 47 | 45 | 43 |
| Example 2 | 36 | 49 | 43 | 44 | 42 |
| Example 3 | 35 | 50 | 44 | 39 | 45 |
| Comparative | 36 | 35 | 36 | 34 | 34 |
| Example 1 | | | | | |
| Comparative | 34 | 42 | 38 | 36 | 37 |
| Example 2 | | | | | |

As can be seen from the results in Table 4 above, the skin moisture content immediately after the application more increased in the case of the makeup cosmetic compositions of Examples 1 to 3 than in those of Comparative Examples 4 and 5, and the increased skin moisturizing ability lasted for 8 hours or more. In the case of the compositions of Comparative Examples 1 to 3, the stability was poor so that the oil was separated and the emulsion was broken, and for these reasons, the skin moisture content could not be measured.

In addition, at the end of the test, a questionnaire survey was performed on the subjects with respect to the effects of the cosmetic compositions on the alleviation of dry skin. The effect was evaluated on a 5-point scale, and the evaluated values (five-point scale) were averaged and shown in Table 5 below.

TABLE 5

| Test material | Effect on alleviation of dry skin |
|---|---|
| Example 1 | 4.4 |
| Example 2 | 4.3 |
| Example 3 | 4.6 |
| Comparative | 1.8 |
| Example 4 | |
| Comparative | 2.1 |
| Example 5 | |

<Criteria for evaluation>
5: very effective.
4: slightly effective.
3: moderate.
2: little effective.
1: not effective.

As can be seen from the results in Table 5 above, the makeup cosmetic compositions of Examples 1 to 3, which contains a large amount of polyol stabilized using the polyglycerin-modified silicone emulsifier according to the present invention, showed better effects on the alleviation of dry skin than the compositions of Comparative Examples 4 and 5.

1. A makeup cosmetic composition containing a polyglycerin-modified dimethione.

2. The makeup cosmetic composition of claim 1, wherein the polyglycerin-modified dimethione is one or more selected from the group consisting of branched polyglycerin-modified dimethiones and crosslinked polyglycerin-modified dimethiones.

3. The makeup cosmetic composition of claim 1, wherein the polyglycerin-modified dimethione is used as an emulsifier.

4. The makeup cosmetic composition of claim 1, wherein the branched or crosslinked polyglycerin-modified dimethione is polyglyceryl-3 disiloxane dimethicone, polyglyceryl-3 polydimethyldisiloxyethyl dimethicone, lauryl polyglyceryl-3 polydimethyldisiloxyethyl dimethicone, dimethicone/polyglycerin-3 crosspolymer or lauryl dimethicone/polyglycerin-3 crosspolymer.

5. The makeup cosmetic composition of claim 1, wherein the composition further contains polyol in an amount of 10-80 wt % based on the total weight of the composition.

6. The water-in-oil makeup cosmetic composition of claim 5, wherein the polyol is one or more selected from the group consisting of glycerin, butylene glycol, propylene glycol, dipropylene glycol, pentylene glycol, hexylene glycol, polyethylene glycol, and sorbitol.

7. The makeup cosmetic composition of claim 2, wherein the polyglycerin-modified dimethione is used as an emulsifier.
